(19) United States
(12) Patent Application Publication
(10) Pub. No.: US 2006/0199280 A1
(43) Pub. Date: Sep. 7, 2006

(54) Quantification of proteins

(76) Inventors: David Bar-Or, Englewood, CO (US); Raphael Bar-Or, Denver, CO (US); Leonard T. Rael, Aurora, CO (US)

Correspondence Address: SHERIDAN ROSS PC, 1560 BROADWAY, SUITE 1200, DENVER, CO, 80202, US

(21) Appl. No.: 11/368,031

(22) Filed: Mar. 3, 2006

Related U.S. Application Data
(60) Provisional application No. 60/658,856, filed on Mar. 3, 2005.

(51) Int. Cl. G01N 33/543

(52) U.S. Cl. 436/518

(57) ABSTRACT

The invention provides a method and kit for determining the quantity of certain proteins comprising post-translationally modified sulfhydryl groups. The invention also provides a method and kit for determining the quantity of a protein having no cysteines in its amino acid sequence.

### CROSS REFERENCE TO RELATED APPLICATION

This application claims priority under 35 U.S.C. § 119(e) to U.S. Provisional Patent Application Ser. No. 60/658,856 filed Mar. 3, 2005, which is incorporated herein in its entirety by this reference.

### FIELD OF THE INVENTION

The invention relates to a method and kit for determining the quantity of proteins that comprise post-translationally modified cysteine sulfhydryl groups. The invention also relates to a method and kit for determining the quantity of proteins that have no cysteines in their amino acid sequence.

### BACKGROUND OF THE INVENTION

Determining the quantity of one or more post-translationally modified proteins can be useful for the diagnosis and monitoring of a variety of diseases and conditions. For instance, PCT application WO 2004/032711 describes methods of diagnosing and monitoring ischemia, inflammation and inflammatory diseases and conditions which utilize measurements of the quantity of certain post-translationally modified proteins. In one preferred embodiment, cysteinylated proteins are measured. PCT application WO 03/001182 describes methods of diagnosing hyperhomocysteinemia and diseases associated with hyperhomocysteinemia (e.g., cardiovascular diseases, coronary artery disease and cerebrovascular diseases) which utilize measurements of the quantity of certain homocysteinylated proteins. In a preferred embodiment, the protein is homocysteinylated transthyretin. U.S. Pat. No. 5,459,076, U.S. Patent Publication No. 2004/0067595 and PCT application WO 98/29452 describe the measurement of S-nitrosylated proteins as being useful in the diagnosis and monitoring a variety of diseases, including inflammation and inflammatory diseases (e.g., asthma and arthritis), sepsis, infections, cardiovascular and cerebrovascular diseases, neurological disorders (e.g., Parkinson's, multiple sclerosis and Alzheimer's disease), ischemia, arthrosclerosis, thrombosis, diabetes, cancer and many others.

Many methods of determining the quantity of post-translationally modified proteins, including cysteinylated and homocysteinylated proteins, are known. See, e.g., the references cited in the previous paragraph. Although these known methods can be used to determine the quantity of post-translationally modified proteins in a biological sample, a need remains for additional methods of determining the quantity of such proteins.

Measurements of apolipoprotein A1 are utilized in the diagnosis of a variety of disease and conditions. Most commonly apolipoprotein A1 measurements are utilized to assess the quantity of high density lipoprotein (HDL) or “good cholesterol” in a patient's blood and to assess inflammation, and assays for apolipoprotein A1 are performed routinely in clinical laboratories. However, a need remains for additional methods of determining the quantity of apolipoprotein A1.

### SUMMARY OF THE INVENTION

The invention provides a method for determining the quantity of a modified-SH protein. The method comprises the following steps: (a) providing a sample comprising one or more free-SH proteins and one or more modified-SH proteins; (b) contacting the sample with a ligand that specifically binds free-SH proteins under conditions effective so that the ligand binds to the free-SH proteins; (c) separating the bound proteins from the unbound proteins to produce a bound fraction comprising the proteins bound to the ligand and an unbound fraction comprising the proteins not bound to the ligand; and (d) determining the quantity of the modified-SH protein in the unbound fraction.

The invention also provides a kit for determining the quantity of a modified-SH protein in a sample. The kit comprises a ligand, which binds specifically to free sulfhydryl groups and instructions for conducting the method of the invention to determine the quantity of a modified-SH protein.

The invention further provides a method for determining the quantity of a no-cys protein. The method comprises the following steps: (a) providing a sample comprising one or more free-SH proteins and one or more no-cys proteins; (b) contacting the sample with a ligand that specifically binds free-SH proteins under conditions effective so that the ligand binds to the free-SH proteins; (c) separating the bound proteins from the unbound proteins to produce a bound fraction comprising the proteins bound to the ligand and an unbound fraction comprising the proteins not bound to the ligand; and (d) determining the quantity of the no-cys protein in the unbound fraction.

The invention also provides a kit for determining the quantity of a no-cys protein in a sample. The kit comprises a ligand, which binds specifically to free sulfhydryl groups and instructions for conducting the method of the invention to determine the quantity of a no-cys protein.

The terms “free-SH protein,” “modified-SH protein,” “no-cys protein” and related terms are defined below.

### BRIEF DESCRIPTION OF THE DRAWINGS

FIG. 1: Mass spectrometer printouts showing, from top to bottom, profiles of human serum albumin extracted from plasma after treatment of the plasma with SULFOLINK® coupling gel at plasma dilutions of 1:50, 1:25, 1:10 and 1:5.

FIG. 2: Mass spectrometer printouts showing, from top to bottom, profiles of human serum albumin extracted from plasma and then treated with SULFOLINK® coupling gel at sample dilutions of 1:10, 1:5 and 1:1, undiluted (neat) sample, and sample untreated with SULFOLINK® coupling gel.

### DETAILED DESCRIPTION OF THE PREFERRED EMBODIMENTS OF THE INVENTION

As used herein, “protein” means protein, polypeptide, oligopeptide, peptide and/or fragments of any of them. Also, the name of a specific protein means the protein and/or fragments of the protein. For instance, “albumin” is used herein to mean the full length protein and/or fragments of albumin. Unless otherwise specified, the name of a specific protein includes all species and sources of such protein. For instance, “albumin” includes albumin from all animals (e.g., bovine albumin, human albumin, etc.) and all tissues and organs known to contain albumin (e.g., serum albumin, urine albumin, etc.) and albumin produced by recombinant DNA techniques.

As used herein, an “all-disulfide protein” means a protein which contains a plurality of cysteines in its amino acid sequence and all of the cysteines are engaged in intramolecular disulfide bonds.

As used herein, a “free-SH protein” means a protein that contains at least one cysteine in its amino acid sequence and at least one cysteine in its amino acid sequence has a free sulfhydryl group.

As used herein, a “free sulfhydryl” means —SH.

As used herein, a “modified-SH protein” means a protein which: (i) contains at least one cysteine in its amino acid sequence, (ii) at least one cysteine in its amino acid sequence has been modified by a post-translational modification of its sulfhydryl group, and (iii) all of the cysteines in its amino acid sequence are either engaged in intramolecular disulfide bonds or have been modified by post-translational modifications of their sulfhydryl groups. If more than one cysteine is modified by a post-translational modification, the post-translational modifications may be the same or different.

As used herein, a “no-cys protein” means a protein that does not contain a cysteine in its amino acid sequence.

As used herein, a “no-free-SH protein” means a protein that does not contain a cysteine in its amino acid sequence which has a free sulfhydryl group. The “no-free-SH proteins” are the all-disulfide proteins, the no-cys proteins and the modified-SH proteins.

As used herein, “post-translational modification” means any modification of a protein that occurs after peptide bond formation. Post-translational modifications of sulfhydryl groups of cysteines include sulfonation, cysteinylation, nitrosylation, homocysteinylation, glutathionylation and glucoronylation.

The invention provides a method for determining the quantity of a modified-SH protein in a sample. Any sample known to contain, or suspected of containing, a modified-SH protein can be used. For instance, the sample can be a body fluid of an animal. Suitable body fluids include blood (e.g., venous blood or cord blood), serum, plasma, urine, saliva, cerebrospinal fluid, tears, semen, vaginal secretions and amniotic fluid. Also, lavages (e.g., bronchial lavages), tissue homogenates and cell lysates can be utilized and, as used herein, the term “body fluid” includes such preparations. The body fluid can be from any animal. Preferably, the animal is a mammal, including humans, dogs, cats, horses, cows, domesticated and farm animals. Most preferably the mammal is a human. As used herein, “patient” is used interchangeably with “animal.” The sample can also be a portion of a protein preparation, such as an albumin preparation, intended for pharmaceutical, research, diagnostic or other uses.

Ligands useful for binding free-SH proteins include antibodies specific for an epitope comprising a free sulfhydryl group on one or more proteins in the sample. Preferably, the antibody is specific for free sulfhydryl groups or for cysteines comprising a free sulfhydryl group so that it will bind to any protein in a sample comprising a free sulfhydryl group. Alternatively, an antibody specific for an epitope on a protein in the sample that comprises a free sulfhydryl group and another portion of the protein unique to that protein so that the antibody will bind specifically to the protein, or a cocktail of such antibodies specific for several different proteins in the sample, can be used. As used in this context, “specific” means that the antibody will bind a free-SH protein(s) selectively in the presence of other proteins and, for some antibodies, will bind a single free-SH protein selectively in the presence of other proteins, including other free-SH proteins.

Antibodies suitable for use in the invention include antisera, polyclonal antibodies, omniclonal antibodies, monoclonal antibodies, bispecific antibodies, humanized antibodies, chimeric antibodies, single-chain antibodies, Fab fragments, F(ab′)₂ fragments, fragments produced by an Fab expression library, epitope-binding fragments of any of the foregoing, and complementarity determining sequences (CDRs). Methods of making antibodies are well known.

Antibodies can be used as the ligand when the free sulfhydryl groups of the protein(s) in the sample are accessible to the antibody, such as where the free sulfhydryl groups are on the surface of the protein(s). Free sulfhydryl groups are not always accessible to antibodies, since antibodies are large molecules. Further, it is not always known whether a free sulfhydryl group of a protein is or is not accessible. Accordingly, ligands which can bind free sulfhydryl groups even when they are not accessible to large molecules, such as antibodies, are preferred. Such ligands include aptamers and coupling agents.

Aptamers can be used in place of, or in combination with, antibodies as ligands. Aptamers are oligonucleotides that are specific for proteins and other non-nucleotide molecules. See, e.g., PCT applications WO 00/70329, WO 01/795692 and WO 99/54506 and U.S. Pat. No. 5,756,291, the complete disclosures of which are incorporated herein by reference. Aptamers suitable for use in the present invention can be prepared using the methods described in these references. Briefly, a heterogeneous population of oligonucleotides of random sequences is synthesized, and a free-SH protein is mixed with the heterogeneous population of oligonucleotides. Complexes are formed with some, but not all, sequences present in the oligonucleotide population. The complexes are isolated and the oligonucleotides recovered and amplified (e.g, by PCR). The resulting mixture of oligonucleotides can be used as the starting material for another round of complexation, isolation and amplification, and the process will typically be repeated several times until an aptamer of satisfactory specificity is obtained and/or until a consensus aptamer sequence is identified.

The most preferred ligands for binding free-SH proteins are coupling agents. A coupling agent is a chemical entity that reacts specifically with free sulfhydryl groups to form a covalent bond. In this context, the term “reacts specifically” and similar terms mean that the coupling agent reacts preferentially with free sulfhydryl groups in the presence of other reactive chemical groups on the protein, such as amino and hydroxyl groups, and does not react with disulfides. Many suitable coupling agents are well known in the art and include maleimide, N-alkyl maleimides (such as N-methyl maleimide, N-ethyl maleimide and N-propyl maleimide), N-alkyl phthalimides, iodoacetate, iodoacetyl, iodoacetamide, iminopyrollidones (such as 4-imino-1,3-diazobicyclo-(3,10)-hexane-2-one), alkane thiosulphonates (such as methane thiosulphonate), fluoro-substituted alkyl phenols (such as 4-trifluoromethyl phenol), and erthopeptidyl epoxides. Methods of using coupling agents to react with free sulfhydryls on proteins are also well known in the art. Those skilled in the art can readily select a suitable coupling agent and will know or can determine suitable conditions for using it.

The free-SH proteins bound to the ligand (the bound fraction) can be separated from the no-free-SH proteins not bound to the ligand (the unbound fraction) in a variety of ways known in the art. Preferably, the ligand is attached to a solid surface to provide a convenient method of separating the bound and unbound fractions. The ligand may be attached directly to the solid surface or may be attached to the solid surface by means of a spacer arm. Methods of attaching antibodies and aptamers to solid surfaces are well known in the art. In the case of coupling agents, the coupling agent is preferably attached to the solid surface by means of a spacer arm of sufficient length (preferably at least 12 atoms in length) to space the coupling agent away from the solid surface so that it can readily reach and react with the free sulfhydryls on the proteins present in a sample. The use of a spacer arm avoids problems of steric hindrance and allows the reaction to proceed more efficiently.

In another preferred embodiment, the free-SH proteins bound to the ligand can be captured by a material attached to a solid surface. For instance, the capture material may be an antibody specific for the sulfhydryls that have reacted with a coupling agent. In such a case, the coupling agent may advantageously be attached to a tag and the antibody will be specific for the tag. For instance, the tag could be the spacer arm. In another alternative, the tag can be biotin and the capture material can be avidin or streptavidin. For instance, EZ-LINk® Maleimide PEO₂-Biotin ((+)-biotinyl-3-maleimidopropionamidyl-3,6-dioxaoctanediamine) (Pierce Biotechnology, Inc., Rockford, Ill.) could be used. The maleimide group of Maleimide PEO₂-Biotin reacts with free sulfhydryl groups at pH 6.5-7.5, the hydrophilic polyethylene oxide (PEO) spacer arm (29.1 Å) imparts water solubility to the molecule, and biotin acts as the tag which would bind to the avidin or streptavidin attached to the solid surface.

Suitable solid surfaces are well known in the art and include plates (e.g., Petri dishes, microtiter plates, etc.), filter paper, substrates (e.g., glass slides, plastic strips), membranes (permeable and impermeable), gels, beads, columns and tubes. Those skilled in the art can readily select a suitable solid surface for use in the present invention.

As a matter of convenience, suitable solid surfaces functionalized with a coupling agent are available commercially. For instance, plates functionalized with maleimide are available from Pierce Biotechnology, Rockford, Ill. (REACTI-BIND™ Maleimide activated plates). Maleimides react with free sulfhydryl groups at a pH of 6.5-7.5, forming stable thioether linkages. An additional product is TNB (5-Thio-2-nitrobenzoic acid)-Thiol Agarose (Pierce Biotechnology, Inc., Rockford, Ill.), which is designed to couple to sulfhydryl containing proteins under mild, nondenaturing conditions.

Another solid surface functionalized with a coupling agent is SULFOLINK® coupling gel (Pierce Biotechnology, Inc., Rockford, Ill.), which is a preferred material for use in practicing the present invention. SULFOLINK® coupling gel is a cross-linked beaded agarose matrix that has been derivatized with 12-atom spacer arms, each of which ends in an iodoacetyl group. The iodoacetyl groups specifically react with free sulfhydryls at pH 7.5-8.5 to form covalent thioether bonds. The spacer arms allow the iodoacetyl groups to react with the free sulfhydryls of proteins that might otherwise be sterically hindered in reacting with a ligand and make binding of free-SH proteins to the gel more efficient.

A similar product, ULTRALINK® Iodoacetyl Gel (Pierce Biotechnology, Inc., Rockford, Ill.), can also be used. ULTRALINK® Iodoacetyl Gel comprises UltraLink® Biosupport that has been derivatized with 15-atom spacer arms, each of which ends in an iodoacetyl group. ULTRALINK® Biosupport is a charge-free, rigid, highly cross-linked, copolymeric and porous resin with high coupling capacity and minimal nonspecific interactions with most sample types. Its porosity, rigidity and durability make it suitable for medium-pressure, fast-flow techniques involving large sample volumes.

The sample and ligand are contacted by any means. Suitable such means are well known in the art and include, for example, mixing, stirring, vortexing, incubating and the like, to allow the ligand to react with the free-SH proteins in the sample. An excess amount of a ligand should be used. An “excess” amount of ligand means an amount of ligand which is greater than that amount which is stoichiometrically required to bind or react with all of the free sulfhydryl groups in a sample. Those skilled in the art can readily determine an appropriate amount of ligand to add to a sample and appropriate conditions (time, temperature, pH, etc.) for contacting the ligand with the sample.

After the ligand has bound to the free-SH proteins in the sample, the free-SH proteins bound to the ligand (bound fraction) are separated from the no-free-SH proteins not bound to the ligand (unbound fraction). Suitable such methods are well known in the art and include centrifugation, settling, washing/eluting a column, etc.

The quantity of one or more of the modified-SH proteins present in the unbound fraction can be determined using a variety of methods, and suitable means of doing so are known in the art, including those described in U.S. Pat. No. 5,459,076, U.S. Patent Publication No. 2004/0067595 and PCT applications WO 98/29452, WO 03/001182, and WO 2004/032711, the complete disclosures of which are incorporated herein by reference. Suitable techniques include mass spectrometry, binding-partner assays and assays which exploit a specific type of post-translational modification. Preferred are binding-partner assays.

Mass spectrometry (MS) can be used to quantitate the modified-SH proteins. The mass of a protein will vary depending on the number and types of post-translational modifications, and the quantities of different modified-SH proteins of different masses can be determined by MS. A single post-translational modification of a single modified-SH protein, two or more post-translational modifications of a single modified-SH protein or post-translational modifications of two or more modified-SH proteins can be quantitated. Indeed, MS provides a way of identifying and quantitating many or all the modified-SH proteins present in a sample or of many or all of the modifications of a single modified-SH protein in a sample. Such MS profiles can be used for diagnosing and monitoring various conditions, diseases and disorders, including inflammation and ischemia.

A variety of MS analysis methods known in the art can be used. For instance, a modified-SH protein can first be isolated from the unbound fraction by any suitable technique known to those skilled in the art, such as liquid chromatography, two-dimensional gel electrophoresis or affinity chromatography. Then, the various post-translational modifications of the sulfhydryl group(s) of the modified-SH protein can be quantitated by any MS detection method, such as electrospray ionization MS (ESI-MS), LCMS, matix-assisted laser desorption/ionization MS (MALDI-MS), MALDI time-of-flight MS (MALDI-TOF-MS), and the like as described in Lim et al., Analytical Biochem, 295:45-56 (2001). One or a plurality of the various post-translational modifications of the sulfhydryl group(s) of the modified-SH protein can be quantitated by, e.g., using standards of pure recombinant proteins, a ratio to the corresponding unmodified protein in the same body fluid, or by comparison to the same protein in the same type of body fluid from normal controls. Percent post-translational modifications can be calculated from total protein species using area under the curve analysis in the resulting mass spectrograms.

Binding-partner assays employ an appropriate binding partner selected for its specificity for a protein of interest. A “binding partner” is any material capable of specifically binding a modified-SH protein remaining in the sample. “Specifically,” “specificity” and the like are used interchangeably herein and mean that the binding partner binds a modified-SH protein selectively in the presence of other proteins, including, in some cases, other modified-SH proteins. For example, the binding partner may have specificity for a portion of the modified-SH protein that includes the post-translationally modified cysteine or for a portion of the modified-SH protein that does not include the post-translationally modified cysteine.

Binding partners include antibodies, aptamers and other proteins and molecules that can bind specifically to a modified-SH protein. Preferred are antibodies and aptamers and binding partner assays utilizing them.

Suitable antibodies are described above, and methods of making antibodies are well known. If desired, a modified-SH protein purified using a ligand as described above can be used as an antigen to produce antibodies specific for epitope(s) containing post-translationally modified cysteine residue(s). Alternatively, a protein or peptide containing a modified cysteine residue can be prepared in vitro and used as the antigen. For example, human serum albumin (HSA) or a peptide corresponding to amino acids 28 to 41 of HSA can be cysteinylated in vitro. Heating the peptide in a slightly alkaline environment in the presence of free cysteine results in cysteinylation of residue Cys34. Verification of protein cysteinylation can be performed by mass spectrometry. Then, the HSA or the peptide conjugated to a carrier protein is used to immunize animals.

A variety of labels and detection methods are known to those skilled in the art. Suitable labels include enzymes, radioactive labels, fluorescent labels, chemiluminescent labels, bioluminescent labels, colorimetric labels, affinity labels, metal colloid labels, latex and silica particles with incorporated dyes and dye particles. The antibodies can be labeled to quantitate the modified-SH proteins or a labeled secondary or tertiary antibody or other antibody-binding compound (e.g., protein A or protein G) can be used to quantitate the modified-SH proteins. Immunoassays can be performed manually or with an automated analyzer.

The antibodies can be used in a variety of immunoassay formats. Suitable immunoassay formats include homogeneous assays, heterogeneous assays, enzyme immunoassays (e.g., ELISA), competitive assays, immunometric (sandwich) assays, turbidimetric assays, nephelometric assays and the like.

Preferred are enzyme immunoassays in which suitable antibodies are immobilized on a solid surface. Suitable solid surfaces are well known and include, for example, glass, glass filters, polystyrene, polypropylene, polyethylene, nylon, polyacrylamide, nitrocellulose, agarose and hydrogel. The immobilized antibody may be, for instance, an antibody specific for an epitope of modified-SH protein which does not contain the post-translationally modified cysteine(s). The sample is contacted with the immobilized antibody so that the modified-SH protein binds to the immobilized antibody. After washing, the modified-SH protein bound to the solid surface by the first antibody is reacted with a second antibody or mixture of antibodies specific for an epitope containing the post-translationally modified cysteine residue(s). The second antibody can be labeled to quantitate the modified-SH protein or a labeled third antibody or other compound that can bind to the second antibody (e.g., protein A or streptavidin) can be used to quantitate the modified-SH protein.

Aptamers can be used in place of, or in combination with, the antibodies in any of the above described assays or in other assays that employ antibodies. Methods of preparing aptamers are described above. Suitable labels for aptamers include dyes, enzymes, radioactive labels, etc.

It is also possible to quantitate modified-SH proteins by liberating the substituents attached to the cysteine residue(s) as a result of the post-translational modification(s) of those residues, and then quantitating either the liberated substituents, the resultant free-SH proteins or both. For instance, the substituents can be liberated using a reducing agent or reducing conditions. Suitable reducing agents and reducing conditions are well known in the art. For instance, the unbound fraction, obtained as described above, could be reduced with dithiothreitol, 2-mercaptoethylamine, mercaptoethanol or tris[2-carboxyethyl]phosphine. Suitable reagents and instructions for their use are available from, e.g., Pierce Biotechnology, Rockford, Ill. The liberated substituent(s), the newly-produced free-SH proteins or both can then be quantitated by a variety of means known in the art, including those assays described above. Also, free sulfhydryl groups can react with a variety of reagents to produce a signal, such as a color signal or a fluorescence signal, which can be measured by methods well known in the art. Suitable such reagents include Ellman's Reagent (5,5-dithio-bis-(2-nitrobenzoic acid)) (available from many sources, including Pierce Biotechnology, Rockford, Ill.) which reacts with free sulfhydryls to produce a distinctive yellow color readable at 412 nm, Thiolyte® Reagents which are essentially nonfluorescent compounds, such as monobromobimane, monochlorobimane, monobromo-trimethylammoniobimane and p-sulfobenzoyloxybromobimane, which are capable of reacting with thiol groups to yield highly fluorescent products (available from Calbiochem, San Diego, Calif.), and DyLight Reactive Fluors which are fluorescent dyes coupled to maleimide (available from Pierce Biotechnology, Rockford, Ill.).

The quantity of one or more modified-SH proteins in a sample can be determined using one of the assays described above. Any method of reporting the quantity of modified-SH proteins may be used. For instance, the quantity may be an amount (e.g., μg) or a concentration (e.g., μM), either of which is typically determined by reference to one or more standards (e.g., a purified recombinant protein that has been post-translationally modified with the same post translational modification(s) of the cysteine(s) as the modified-SH proteins being assayed). The quantity may also be a ratio or percentage compared to another compound, such as the corresponding free-SH protein in the same sample, the same modified-SH protein in the same type of sample from a normal patient, or compared to the total protein in the sample, provided the total protein is determined prior to separating out the modified-SH proteins. Total protein measurements can be made by any means known in the art.

The method of the present invention is useful for a variety of applications. The method can be used for clinical diagnosis and monitoring of diseases, disorders and conditions. Once the quantity of a modified-SH protein in a sample is determined, then a comparison is made to determine if the quantity is significantly altered compared to its level in the same type of sample from normal animals. If so, then the presence of a disease, disorder or condition is indicated. As used herein, “normal animals” are healthy animals who are not suffering from a particular disease, disorder or condition to be diagnosed or monitored. “Significantly” means statistically significant. Suitable methods of statistical analysis are well known in the art. “Altered” means any change or combination of changes in the level of one or more modified-SH proteins and/or in the type of post-translational modification(s) of the cysteines of the modified-SH protein(s). For example, a cysteine of a protein may be post-translationally modified for the first time, the level of a particular post-translational modification of one or more cysteine residues may be increased, decreased or eliminated, etc.

The method can be used in the clinical diagnosis, assessment and monitoring of any disease, disorder or condition in which one or more proteins has been post-translationally modified on its cysteine sulfhydryl groups. Such diseases, disorders and conditions include ischemia (e.g., cardiac, bowel and placental ischemia), inflammation, inflammatory diseases, disorders and conditions (e.g., adult respiratory distress syndrome, allergies, arthritis, asthma, autoimmune diseases (e.g., multiple sclerosis), bronchitis, cancer, cardiovascular disease, chronic obstructive pulmonary disease, Crohn's disease, cystic fibrosis, emphysema, endocarditis, gastritis, graft-versus-host disease, infections (e.g., bacterial, viral and parasitic), inflammatory bowel disease, injuries, ischemia (e.g., heart, brain, bowel and placental), multiple organ dysfunction syndrome (multiple organ failure), nephritis, neurodegenerative diseases (e.g., Alzheimer's disease and Parkinson's disease), ophthalmic inflammation, pain, pancreatitis, psoriasis, sepsis, shock, transplant rejection, trauma, ulcers (e.g., gastrointestinal ulcers and ulcerative colitis), etc.), cardiovascular diseases, coronary artery disease, cerebrovascular diseases, preeclampsia, fetal growth restriction, neurological ailments, cognitive dysfunction, renal disease and diabetes. The modified-SH proteins that can be measured include:
1. Homocysteinylated transthyretin, homocysteinylated fibronectin and homocysteinylated albumin for the diagnosis of hyperhomocysteinemia and diseases, disorders and conditions associated with it, including cardiovascular diseases, coronary artery disease, cerebrovascular diseases, preeclampsia, fetal growth restriction, neurological ailments, cognitive dysfunction, renal disease and diabetes.
2. Cysteinylated blood proteins, including albumin, for the diagnosis of ischemia.
3. Cysteinylated tissue-specific, organ-specific and disease-specific proteins, including cardiac troponin I, cardiac troponin T, creatinine phosphokinase, its MB isoenzyme, myoglobin, an S100 protein, enolase, β-human chorionic gonadotropin, α-fetoprotein, pregnancy-associated protein 1A, erythropoietin and angiotensin, for the diagnosis of specific types of ischemia.
4. Cysteinylated blood proteins, including albumin, immunoglobulins and C-reactive protein, for the diagnosis of inflammation and inflammatory diseases, disorders and conditions.
5. S-Nitrosylated blood proteins, including albumin, immunoglobulins and C-reactive protein, for the diagnosis of inflammation and inflammatory diseases, disorders and conditions.
6. Cysteinylated tissue-specific, organ-specific and disease-specific proteins, including cardiac troponin I, cardiac troponin T, creatinine phosphokinase, its MB isoenzyme, myoglobin, an S100 protein, enolase, β-human chorionic gonadotropin, α-fetoprotein, pregnancy-associated protein 1A, erythropoietin, angiotensin, β-amyloid, α-synuclein, myelin basic protein, liver enzymes, brca1, cea, psa, α1-antitrypsin, surfactant proteins, elastase, Rheumatoid factor, collagen and lipopolysaccharide binding proteins, for the diagnosis of specific types of inflammation and specific inflammatory diseases, disorders and conditions.
7. S-Nitrosylated tissue-specific, organ-specific and disease-specific proteins, including cardiac troponin I, cardiac troponin T, creatinine phosphokinase, its MB isoenzyme, myoglobin, an S100 protein, enolase, β-human chorionic gonadotropin, α-fetoprotein, pregnancy-associated protein 1A, erythropoietin, angiotensin, β-amyloid, α-synuclein, myelin basic protein, liver enzymes, brca1, cea, psa, α1-antitrypsin, surfactant proteins, elastase, Rheumatoid factor, collagen and lipopolysaccharide binding proteins, for the diagnosis of specific types of inflammation and specific inflammatory diseases, disorders and conditions.
8. Cysteinylated blood proteins, including albumin, for the diagnosis of multiple organ failure.
9. Cysteinylated proteins, including albumin, β-human chorionic gonadotropin, α-fetoprotein, pregnancy-associated protein 1A, erythropoietin, angiotensin and other pregnancy-associated proteins, for the diagnosis of placental ischemia, preeclampsia and fetal growth retardation.
10. All modified-SH forms of albumin for the diagnosis of inflammation and the oxidative status of a patient.

The method of the invention can also be used to determine and monitor the quantity of modified-SH protein present in a protein preparation, such as those to be used in therapeutic, research, diagnostic or other applications. The quantity of modified-SH protein can be monitored before and/or after one or more steps of the process used to manufacture the protein preparation and/or at the end of the process (i.e., the quantity of modified-SH protein present in the final protein preparation). Thus, the method of the invention can be used as part of the quality control of manufacturing processes, for standardization of protein preparations with respect to their content of modified-SH protein (see below), and for monitoring protein preparations prior to their use (e.g., determining the quantity of modified-SH protein in a preparation before administering the preparation to a patient).

In particular, it would be highly desirable to have protein preparations for therapeutic, research, diagnostic and other uses that contain known amounts of modified-SH protein that are suitable for an intended application. Accordingly, the method of the present invention can further include the step determining whether the quantity of a modified-SH protein is an acceptable quantity for a desired application. For example, the acceptable quantity for therapeutic applications would be a therapeutically-acceptable quantity. Such therapeutically-acceptable quantity can be the quantity found in normal patients or another quantity predetermined by a skilled clinician. Similarly, those skilled in the art can readily determine an appropriate quantity for a research, diagnostic or other application.

The quantity of modified-SH protein in a protein preparation can be adjusted to an acceptable or desired quantity in a variety of ways. For instance, all of the steps of a manufacturing process can be monitored by measuring the quantity of modified-SH protein before and after each step, and steps that cause the production of modified-SH protein can be modified or replaced. In addition or alternatively, the quantity of modified-SH protein can be reduced by removing some or all of the modified-SH protein from the protein preparation, preferably as the last step or one of the last steps of the manufacturing process. For instance, the quantity of modified-SH protein can be reduced using affinity chromatography. This can be accomplished using a column of beads having attached thereto an antibody or antibodies specific for one or more modified-SH proteins to remove modified-SH proteins from the protein preparation. Alternatively, a column of beads having attached thereto an antibody or antibodies specific for the free-SH protein. The modified-SH proteins pass through the column, after which the free-SH proteins are eluted from the column. The eluate containing the free-SH proteins can be used as eluted from the column or a certain portion of the eluate containing the modified-SH proteins can be added to the eluate containing the free-SH protein to obtain an acceptable or desire quantity of modified-SH proteins. The quantity of modified-SH protein present in the protein preparation (the final preparation and at one or more stages of the manufacturing process) can be monitored using the method of the present invention or another method, including known prior art methods (e.g., mass spectrometry).

Thus, the invention can also provide protein preparations containing a known amount of modified-SH protein, including protein preparations containing acceptable or desired amounts of modified-SH protein. The protein preparations will be provided in a container, and the container will have a label on or associated with it stating the amount of modified-SH protein in the protein preparation. The protein preparation may be plasma, an immunoglobulin preparation or an erythropoietin preparation.

The invention also provides kits for determining the quantity of a modified-SH protein in a sample. The kits can be formatted for use in a diagnostic apparatus (e.g., an automated analyzer) or can be self-contained (e.g., for a point-of-care diagnostic). The kits will contain a ligand according to the present invention. The ligand is preferably a coupling agent, and the ligand is preferably attached to a solid surface as described above. The kits can also contain additional reagents and components useful in performing the methods of the invention. The ligands and other reagents and components will be held in suitable containers, which include bottles, vials, test tubes, microtiter plates, boxes and bags (e.g., bags made of paper, foil or cellophane). Reagents, such as binding partners, can be incorporated into or onto substrates, test strips made of filter paper, glass, metal, plastics or gels and other devices suitable for performing binding partner assays. Instructions for performing the methods of the present invention will also be provided. The kits can also contain other useful associated materials that are known in the art and that may be desirable from a commercial or user standpoint, such as buffers, enzyme substrates, diluents, standards and the like. Finally, the kits can also include containers for performing the methods, for collecting, diluting and/or measuring a sample and/or reagents.

In preferred embodiments of the invention, the quantities of modified-SH albumins are measured. It has been shown that the quantity of cysteinylated albumin is increased in inflammation and ischemia. See PCT application WO 2004/032711. The quantity of all species of modified-SH albumins will provide a measure of the oxidant status and capacity of a patient's blood, and this quantity may provide an indication of a patient's outcome in cases of serious illnesses. The quantity of nitrosylated albumin is increased in inflammation, and the level of homocysteinylated albumin is increased in hyperhomocysteinemia and diseases associated with hyperhomocysteinemia (see PCT application WO 03/001182). The quantities of these modified-SH albumins can be measured as described herein to diagnose and monitor these diseases and conditions.

Albumin therapeutic preparations are used for the treatment of shock, urgent restoration of blood volume, acute management of burns, and hypoalbuminemia. However, conflicting reports exist in the literature that question the clinical safety and efficacy of human serum albumin (HSA) when administered to clinically ill patients. It has recently been shown that currently available commercial HSA preparations have significantly higher levels of modified-SH albumins, including significantly higher levels of S-nitrosylated albumin, compared to normal plasma, and that the levels of modified-SH albumins vary from one manufacturer to another and suffer from lot-to-lot variability in lots from the same manufacturer. Bar-Or, D., Bar-Or, R., Rael, L. T., Gardner, D, Slone, D. S. and Craun, M. L., “Heterogeneity And Oxidation Status Of Commercial Human Albumin Preparations In Clinical Use,” submitted for publication. See also, Gryzunov et al., Arch. Biochem. Biophys., 413:53-66 (2003). While not being bound by any particular theory, it is believed that oxidized forms of HSA might augment oxidative stress when administered to patients for whom HSA is clinically indicated. In addition, the antioxidant potential of commercially available albumin preparations is diminished by the oxidation of albumin during its preparation.

Albumin is also a common reagent used in research, diagnostics and culturing cells. For instance, HSA is a major component in in vitro fertilization media, and controlling the content of oxidized albumin would be important to prevent unnecessary oxidative stress on the reproductive cells during the fertilization process.

Accordingly, being able to determine, know and/or adjust the quantity of modified-SH albumins in an albumin preparation is clearly needed. The invention provides a method of quantitating modified-SH albumins. In addition, albumin preparations containing an acceptable or desired quantity of modified-SH albumin can be prepared as described above using, e.g., affinity chromatography to prepare such albumin preparations.

As can be readily appreciated, the unbound fraction, obtained as described above, may comprise no-cys proteins in addition to modified-SH proteins, and the invention also provides a method of determining the quantity of a no-cys protein in a sample that contains or is suspected of containing such a protein. The samples include those described above. Preferred is a plasma or serum sample. A no-cys protein in the unbound fraction can be quantitated by mass spectrometry as described above. A no-cys protein in the unbound fraction can also be quantitated by means of a binding partner assay as described above for the modified-SH proteins, but using a binding partner specific for the no-cys protein. One such no-cys protein is apolipoprotein A1.

### EXAMPLES

The following Examples are intended to illustrate embodiments of the invention and are not intended to limit the invention.

### Example 1

### Characterization of Modified-SH Albumin Species in Human Plasma

A. Isolation Of Modified-SH Albumin From Human Plasma

The following protocol was used to isolate modified-SH albumin from human plasma:
1. Whole blood was obtained from a healthy volunteer by venopuncture into heparin-containing Vacutainer tubes. The tubes were spun for 10 minutes at 1500 rpm. The plasma was collected, aliquoted, and stored at −80° C.
2. For each plasma sample (see step 3), 300 μL of SULFOLINK® Coupling Gel (Pierce Biotechnology, Rockford, Ill.) was added to a 1.7 mL microcentrifuge tube. The gel was equilibrated according to the manufacturer's protocol with 0.5 mL of coupling buffer (50 mM Tris, 5 mM Na-EDTA, pH 8.5), vortexed, and centrifuged at top speed for 2 seconds. The supernatant was discarded, and the equilibration process was repeated 2 more times.
3. Next, 150 μL of a 1:5 dilution of human plasma in coupling buffer was added to the tube containing the equilibrated SULFOLINK® gel. The tube was vortexed and mixed end-over-end at room temperature for 30 minutes, and then it was incubated an additional 15 minutes without mixing at room temperature.
4. The tube was centrifuged at top speed for 2 seconds. The supernatant was collected and then processed and analyzed by liquid chromatography followed by mass spectrometry (LCMS) as described in sections B and C below for the presence of various albumin species. A sample of plasma diluted 1:5 that was not treated with SULFOLINK® gel was also processed and analyzed by LCMS for comparison purposes. Finally, total protein was measured as described in section D below before and after SULFOLINK® gel treatment to assess the efficiency of free-SH albumin removal.

B. Isolation of Albumin from Plasma

Prior to performing LCMS, albumin was extracted as follows:
1. Place one SwellGel Blue Albumin Removal Disc (Pierce Biotechnology, Rockford, Ill.) into a Mini-Spin column (Pierce Biotechnology, Rockford, Ill.). Hydrate the disc with 380 μL of ultrapure water. Place the column into a 1.7 mL microcentrifuge tube and centrifuge at 10,000 rpm for 1 minute. Discard the flow-through.
2. Load 100 μL of the supernatant from step 4 of section A or 100 μL of the 1:5 diluted plasma from step 3 of section A onto the disc in the column and incubate for 2 minutes. Centrifuge the column at 10,000 rpm for 1 minute. Re-apply the flow-through to the column and incubate 2 minutes. Centrifuge the column at 10,000 rpm for 1 minute. Discard the flow-through.
3. Wash the column to remove unbound proteins by adding 50 μL Binding Wash Buffer (Pierce Biotechnology, Rockford, Ill.) to the disc. Centrifuge the column at 10,000 rpm for 1 minute. Repeat the wash step three more times. Discard the tube and use a new collection tube.
4. Add 200 μL of 1 M NaCl to the column. Centrifuge at 10,000 rpm for 1 minute. Retain the flow-through. Repeat the elution step three more times with 200 μL of 1 M NaCl.
5. De-salt the retained flow-through (eluate) on a Microcon 30 spin column (Pierce Biotechnology, Rockford, Ill.). Add 500 μL of eluate to the column and centrifuge at 8500 rpm for 8 minutes. Discard the flow-through. Add the remainder of the eluate and repeat the centrifugation. Discard the flow-through.
6. Rinse the column, which contains proteins >30 kDa, 3 times with 300 μL 18 mΩ water. Discard the flow-through each time.
7. Invert the filter from the Microcon 30 in a microcentrifuge tube and spin at 3000 rpm for 2 minutes to obtain a solution containing proteins >30 kDa (the >30 kDa fraction). The collected >30 kDa fraction was analyzed by LCMS as described in section C below.

C. LCMS Analysis of Albumin Species

LCMS analysis of albumin species was performed as follows:
1. The >30 kDa fractions from step 7 of section B obtained by processing the supernatant from step 4 of section A and the 1:5 diluted plasma from step 3 of section A were injected onto a YMC-Pack Protein-RP HPLC column (Waters, Milford, Mass., USA) using a linear gradient system of:
   A. water/0.1% trifluoroacetic acid (TFA)
   B. acetonitrile/0.1% TFA
      The linear gradient starts at 100% A and goes to 80% B in 20 minutes using a Waters 2795 HPLC system (Waters, Milford, Mass., USA).
2. Mass spectrometry was performed using a time of flight (TOF) mass spectrometer (Micromass LCT, UK) run in positive electrospray ionization mode at +30 ev and desolvation temperature of 200° C. The spectra were deconvolved using MaxEnt I. Percent cysteinylated albumin was calculated from total albumin species using area under the curve analysis in the resulting mass spectrogram.

D. Total Protein

Total protein was measured by Coomassie Protein Assay, Pierce Biotechnology, Rockford, Ill.

E. Results

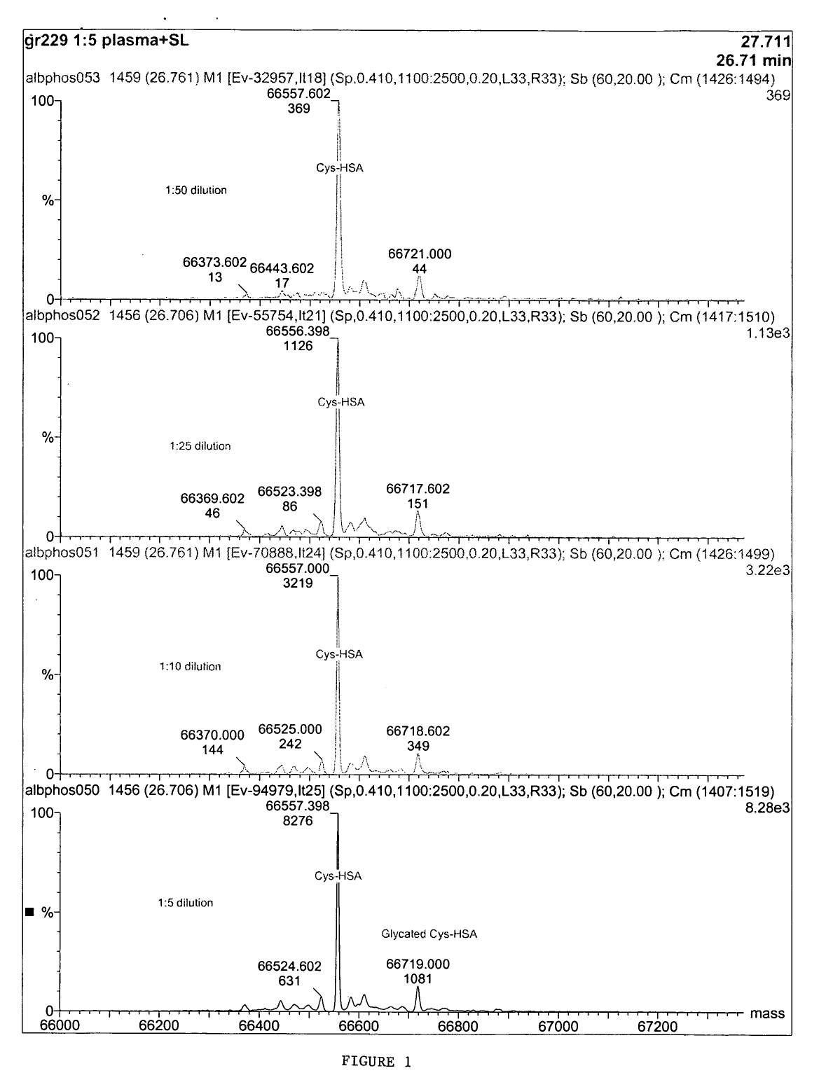

The LCMS results are shown in FIG. 1. As can be seen from FIG. 1, plasma after treatment with SULFOLINK® gel contains a very large amount of cysteinylated albumin and no detectable free-SH (native) albumin

The total protein results are shown in Table 1 below. As can be seen from Table 1, the SULFOLINK® gel removed a substantial amount of protein at all dilutions.

TABLE 1

| | % Decrease in [Total Protein] |
|---|---|
| Sample | as a result of SULFOLINK ® gel |
| 1:5 Dilution | 79.7 |
| 1:10 Dilution | 84.9 |
| 1:25 Dilution | 79.4 |
| 1:50 Dilution | 85.8 |

### Example 2

### Characterization of Modified-SH Albumin Species

Albumin was extracted from plasma as described in Example 1, section B, except that 100 μL of undiluted plasma was used in step 2. Generally, albumin isolated in this fashion generates an albumin concentration of about 7 mg/mL.

A sample of the extracted albumin was diluted 1:1 in coupling buffer and treated with the SULFOLINK® Coupling Gel as described in Example 1, section A. The resulting supernatant was collected and analyzed by LCMS as described in Example 1, section C for the presence of various albumin species. Additionally, the 1:1 dilution that was not treated with SULFOLINK® Coupling Gel was analyzed by LCMS for comparison purposes.

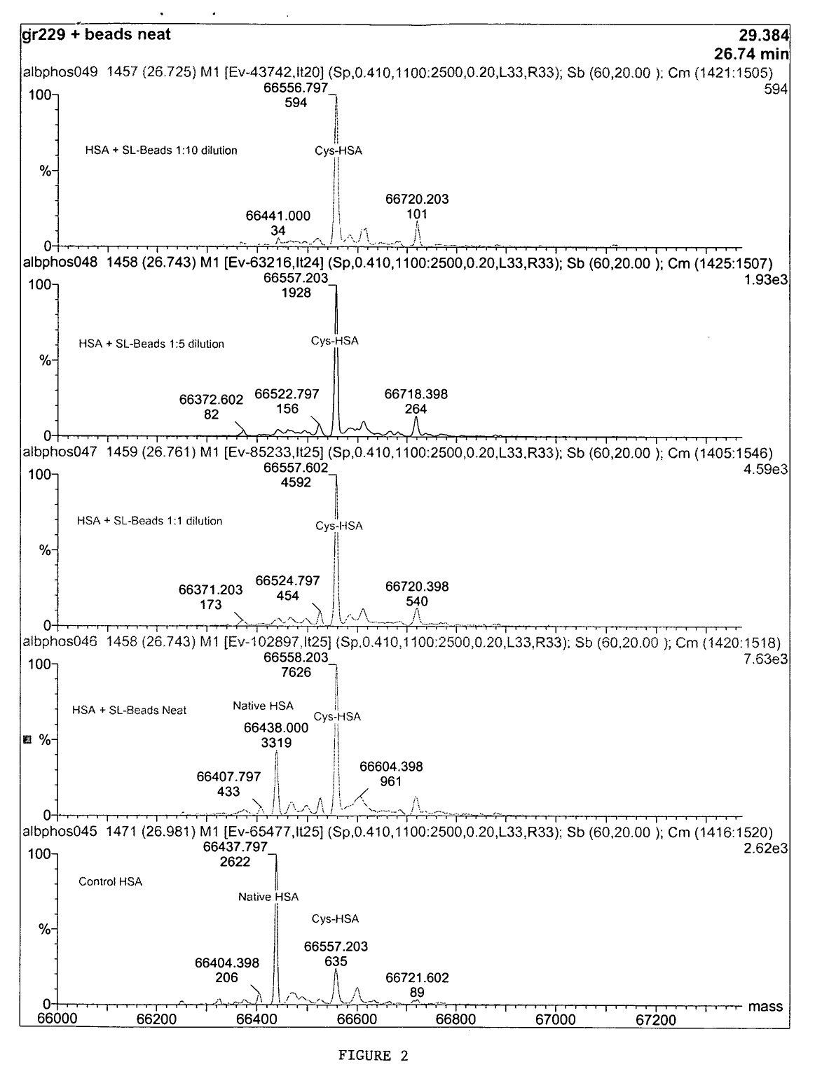

The results are shown in FIG. 2. As can be seen from FIG. 2, treatment with SULFOLINK® gel removed free-SH (native) albumin and increased the amount of cysteinylated albumin present in the SULFOLINK® gel eluate in a dose dependent manner.

The above description of the invention, including the Examples, is intended to be merely illustrative of the invention and is not intended to limit the invention. Numerous variations, modifications and changes can be made by those skilled in the art in light of the above description without departing from the spirit and scope of the invention.

What is claimed is:

1. A method for determining the quantity of a modified-SH protein comprising:
(a) providing a sample comprising one or more free-SH proteins and one or more modified-SH proteins;
(b) contacting the sample with a ligand that specifically binds free-SH proteins under conditions effective so that the ligand binds to the free-SH proteins;
(c) separating the bound proteins from the unbound proteins to produce a bound fraction comprising the proteins bound to the ligand and an unbound fraction comprising the proteins not bound to the ligand; and
(d) determining the quantity of the modified-SH protein in the unbound fraction.

2. The method of claim 1 wherein the ligand is a coupling agent having specific reactivity with free sulfhydryl groups.

3. The method of claim 2 wherein the coupling agent comprises a solid surface having attached thereto a plurality of spacer arms, each spacer arm having attached to it, at or near the end of the spacer arm which is not attached to the solid surface, a chemical entity reactive with free sulfhydryl groups.

4. The method of claim 3 wherein the solid surface is a cross-linked beaded agarose matrix and the chemical entity is an iodoacetyl group.

5. The method of claim 1 wherein the modified-SH protein is a cysteinylated protein, a nitrosylated protein, a homocysteinylated protein, a glutathionylated protein, a sulfonated protein, a glucoronylated protein, or a combination of two or more of the foregoing.

6. The method of claim 1 wherein the modified-SH protein is a cysteinylated protein.

7. The method of claim 1 wherein the modified-SH protein is an organ-specific, tissue-specific or disease-specific protein.

8. The method of claim 1 wherein the modified-SH protein is a blood protein.

9. The method of claim 1 wherein the modified-SH protein is cardiac troponin I or cardiac troponin T.

10. The method of claim 1 wherein the modified-SH protein is transthyretin.

11. The method of claim 1 wherein the sample is a body fluid from an animal.

12. The method of claim 11 wherein animal is human.

13. The method of claim 11 or 12 wherein the body fluid is blood, serum, plasma, urine, saliva, cerebrospinal fluid, tears, semen, vaginal secretion, amniotic fluid, cord blood, lavage, tissue homogenate or cell lysate.

14. The method of claim 13 wherein the body fluid is urine, serum or plasma.

15. The method of claim 1 wherein the quantity of the modified-SH protein is determined by a binding partner assay.

16. The method of claim 15 wherein the binding partner is an aptamer.

17. The method of claim 15 wherein the binding partner is an antibody.

18. The method of claim 18 wherein the antibody is a monoclonal antibody.

19. The method of claim 1 wherein the quantity of the modified-SH protein is determined by contacting the unbound fraction with a reducing agent to release substituents bound to the sulfhydryl groups of the protein and measuring the quantity of the released substituents, the quantity of the resulting free-SH proteins or both.

20. The method of claim 1 wherein the sample is a protein preparation.

21. The method of claim 1 further comprising the step of:
(e) determining if the quantity of the modified-SH protein in the sample is an acceptable quantity for a desired application.

22. The method of claim 21 further comprising the step of:
(f) adjusting the quantity of the modified-SH protein in a sample that does not contain an acceptable quantity to an acceptable quantity.

23. A method for determining the quantity of a modified-SH albumin comprising:
(a) providing a sample comprising free-SH albumin and modified-SH albumin;
(b) contacting the sample with a ligand that specifically binds proteins having a free sulfhydryl group under conditions effective so that the ligand binds to the free-SH albumin;
(c) separating the bound albumin from the unbound albumin to produce a bound fraction comprising the albumin bound to the ligand and an unbound fraction containing the albumin not bound to the ligand; and
(d) determining the quantity of the modified-SH albumin in the unbound fraction.

24. The method of claim 23 wherein the ligand is a coupling agent having specific reactivity with free sulfhydryl groups.

25. The method of claim 24 wherein the coupling agent comprises a solid surface having attached thereto a plurality of spacer arms, each spacer arm having attached to it, at or near the end of the spacer arm which is not attached to the solid surface, a chemical entity reactive with free sulfhydryl groups.

26. The method of claim 25 wherein the solid surface is a cross-linked beaded agarose matrix and the chemical entity is an iodoacetyl group.

27. The method of claim 23 wherein the modified-SH albumin is a cysteinylated albumin, a nitrosylated albumin, a homocysteinyled albumin, a glutathionylated albumin, a sulfonated albumin, a glucoronylated albumin, or a combination of two or more of the foregoing.

28. The method of claim 23 wherein the sample is a body fluid from an animal.

29. The method of claim 28 wherein animal is human.

30. The method of claim 28 or 29 wherein the body fluid is blood, serum, plasma urine, saliva, cerebrospinal fluid, tears, semen, vaginal secretion, amniotic fluid, cord blood, lavage, tissue homogenate or cell lysate.

31. The method of claim 30 wherein the body fluid is urine, serum or plasma.

32. The method of claim 23 wherein the quantity of the modified-SH albumin is determined by a binding partner assay.

33. The method of claim 32 wherein the binding partner binds specifically to an epitope present on all forms of modified-SH albumin.

34. The method of claim 32 wherein the binding partner binds specifically to a single type of modified-SH albumin.

35. The method of claim 34 wherein the binding partner binds specifically to cysteinylated albumin.

36. The method of any one of claims 32-35 wherein the binding partner is an antibody.

37. The method of claim 36 wherein the antibody is a monoclonal antibody.

38. The method of any one of claims 32-35 wherein the binding partner is an aptamer.

39. The method of claim 23 wherein the quantity of the modified-SH albumin is determined by contacting the unbound fraction with a reducing agent to release substituents bound to the sulfhydryl group of the albumin and measuring the quantity of the released substituents, the free-SH albumin or both.

40. The method of claim 23 further comprising the step of:
(e) determining if the quantity of the modified-SH albumin in the sample is an acceptable quantity for a desired application.

41. The method of claim 40 further comprising the step of:
(f) adjusting the quantity of the modified-SH albumin in a sample that does not contain an acceptable quantity to an acceptable quantity.

42. A kit for determining the quantity of a modified-SH protein in a sample comprising a ligand which binds specifically to free sulfhydryl groups and instructions for conducting the method of claim 1.

43. The kit of claim 42 wherein the ligand is a coupling agent having specific reactivity with free sulfhydryl groups.

44. The kit of claim 43 wherein the coupling agent comprises a solid surface having attached thereto a plurality of spacer arms, each spacer arm having attached to it, at or near the end of the spacer arm which is not attached to the solid surface, a chemical entity reactive with free sulfhydryl groups.

45. The kit of claim 44 wherein the solid surface is a cross-linked beaded agarose matrix and the chemical entity is an iodoacetyl group.

46. The kit of any one of claims 42-45 further comprising a binding partner.

47. The kit of claim 46 wherein the binding partner binds specifically to an epitope present in all forms of a modified-SH protein.

48. The kit of claim 46 wherein the binding partner binds specifically to a single type of a modified-SH protein.

49. The kit of claim 48 wherein the binding partner binds specifically to a cysteinylated protein.

50. The kit of any one of claims 46-49 wherein the binding partner is an aptamer.

51. The kit of any one of claims 46-49 wherein the binding partner is an antibody.

52. The kit of claim 51 wherein the binding partner is a monoclonal antibody.

53. A method for determining the quantity of a no-cys protein comprising:
(a) providing a sample comprising one or more free-SH proteins and one or more no-cys proteins;
(b) contacting the sample with a ligand that specifically binds free-SH proteins under conditions effective so that the ligand binds to the free-SH proteins;
(c) separating the bound proteins from the unbound proteins to produce a bound fraction comprising the proteins bound to the ligand and an unbound fraction comprising the proteins not bound to the ligand; and
(d) determining the quantity of the no-cys protein in the unbound fraction.

54. The method of claim 53 wherein the ligand is a coupling agent having specific reactivity with free sulfhydryl groups.

55. The method of claim 54 wherein the coupling agent comprises a solid surface having attached thereto a plurality of spacer arms, each spacer arm having attached to it, at or near the end of the spacer arm which is not attached to the solid surface, a chemical entity reactive with free sulfhydryl groups.

56. The method of claim 55 wherein the solid surface is a cross-linked beaded agarose matrix and the chemical entity is an iodoacetyl group.

57. The method of claim 53 wherein the no-cys protein is apolipoprotein A1.

58. The method of claim 53 wherein the sample is a body fluid from an animal.

59. The method of claim 58 wherein animal is human.

60. The method of claim 58 or 59 wherein the body fluid is blood, serum or plasma.

61. The method of claim 53 wherein the quantity of the no-cys protein is determined by a binding partner assay.

62. The method of claim 61 wherein the binding partner is an aptamer.

63. The method of claim 61 wherein the binding partner is an antibody.

64. The method of claim 63 wherein the antibody is a monoclonal antibody.

65. A kit for determining the quantity of a no-cys protein in a sample comprising a ligand which binds specifically to free sulfhydryl groups and instructions for conducting the method of any one of claims 53-64.

66. The kit of claim 65 wherein the ligand is a coupling agent having specific reactivity with free sulfhydryl groups.

67. The kit of claim 66 wherein the coupling agent comprises a solid surface having attached thereto a plurality of spacer arms, each spacer arm having attached to it, at or near the end of the spacer arm which is not attached to the solid surface, a chemical entity reactive with free sulfhydryl groups.

68. The kit of claim 67 wherein the solid surface is a cross-linked beaded agarose matrix and the chemical entity is an iodoacetyl group.

69. The kit of any one of claims 65-68 further comprising a binding partner.

70. The kit of claim 69 wherein the binding partner is an aptamer.

71. The kit of claim 69 wherein the binding partner is an antibody.

72. The kit of claim 71 wherein the binding partner is a monoclonal antibody.
